(19) United States
(12) Patent Application Publication
(10) Pub. No.: US 2008/0130972 A1
(43) Pub. Date: Jun. 5, 2008

(54) STORING IMAGING PARAMETERS

(75) Inventors: Steven Charles Miller, Pewaukee, WI (US); Patrick Robert Meyers, Mequon, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/565,409

(22) Filed: Nov. 30, 2006

(51) Int. Cl. G06K 9/00; A61B 5/00

(52) U.S. Cl. 382/131; 705/3

(57) ABSTRACT

A method of imaging. The method includes determining imaging parameters. Further, the method includes acquiring image data from a patient. Additionally, the method includes storing the imaging parameters with the acquired image data. The method also includes retrieving the stored imaging parameters for use in a subsequent examination. Systems and computer-readable medium that afford functionality of the type defined by this method are also contemplated in conjunction with the present technique.

### BACKGROUND

The invention relates generally to reviewing medical imaging exams, and more particularly, to reviewing interval changes occurring between two or more images corresponding to a given patient, where the images are produced at different points in time.

Diagnostic imaging has emerged into an essential aspect of patient care. For example, diagnostic imaging devices are employed for detecting and following evolution of disease states, like lesions, that may lead to potential cancers. Furthermore, diagnostic imaging devices are also utilized to monitor the effect of treatment on the disease states.

In traditional approaches for the diagnosis of disease states, and more generally of medical conditions or events, a clinician typically considers an image representative of a region of interest of the patient to discern characteristic features of interest. In cardiac imaging, such features may include coronary arteries or stenotic lesions of interest, and other features, which would be discernable in the image, based upon the skill and knowledge of the individual clinician. Other analyses may be based upon capabilities of various algorithms, including algorithms generally referred to as computer-aided detection or computer-aided diagnosis (CAD) algorithms.

Also, in clinical situations such as serial studies, medical images, such as X-ray images or ultrasound images, for example, obtained at a current examination, are typically compared with a corresponding previously produced image that has been acquired at a previous examination. This comparison of temporally sequential images aids the clinician in identifying abnormalities and determining their significance. Additionally, any interval changes in known abnormalities, such as lesions, may also be studied to determine the efficacy of treatment. As used herein, an interval change may be defined as a pathological change that has occurred after a previous examination and before a current examination.

As will be appreciated, ultrasound imaging, such as intravascular ultrasound provides excellent resolution of structures within a vessel, thereby allowing enhanced assessment of vessels that are often difficult to assess angiographically. However, detection of interval changes between two serial ultrasound images is dependent upon the knowledge and understanding of potential ultrasonic artifacts of the clinician, which may adversely affect image quality, increase the difficulty of image interpretation, or reduce the accuracy of quantitative measurements. Additionally, computed tomography (CT) imaging advantageously provides a description of anatomy in great detail and consequently is being increasingly used for detecting and following the evolution of lesions that may lead to potential cancers. However, a considerable amount of information is presented to the clinician for use in interpreting the images and detecting suspect regions that may indicate disease, thereby resulting in a time-consuming and tedious process. This overload of image data for interpretation may disadvantageously lead to missed detection, as it is difficult to identify a suspicious area in an extensive amount of data. Further, due to the difficulty in comparing two serial X-ray images by scanning back and forth between the two X-ray images, and also due to differences in density, contrast or patient positioning between the two radiographs, important interval changes may be overlooked by clinicians. Additionally, interval changes of a patient having a number of abnormalities often can be missed because some abnormalities are camouflaged by other abnormalities not showing any change.

Furthermore, in general, subsequent images, such as temporally sequential images, are difficult to reproduce in terms of imaging conditions, such as patient positioning, X-ray projection, and other exposure conditions, to name a few. Also, respiration and cardiac pulsation of a patient is typically at different phases for the two images, thereby resulting in changes in the size and the shape of anatomical regions, such as the lungs, the diaphragm and the heart. More particularly, with ultrasound imaging, the availability of a relatively large number of user adjustable imaging parameters disadvantageously leads to difficulty in consistently repeating the imaging conditions. Consequently, there exists an unpredictable change between the temporally sequential images. This unpredictable change may disadvantageously result in missed detection and/or diagnosis. Further, currently available systems are not configured to adapt their acquisition and processing protocols based on predetermined settings. In other words, the current systems are not configured to permit customization of imaging parameters based on a patient and/or predetermined settings.

It may therefore be desirable to develop a robust technique and system for processing image data that advantageously facilitates substantially superior serial study acquisition. In particular, there is a need for a system that can aid in enhancing ease of obtaining information related to interval changes in temporally sequential images of a patient thereby improving the possibilities of detecting important changes in pathology.

### BRIEF DESCRIPTION

In accordance with aspects of the present technique, a method of imaging is presented. The method includes determining imaging parameters. Further, the method includes acquiring image data from a patient. Additionally, the method includes storing the imaging parameters with the acquired image data. The method also includes retrieving the stored imaging parameters for use in a subsequent examination.

In accordance with another aspect of the present technique, a method of imaging is presented. The method includes acquiring a current image data set from a patient based on predetermined imaging parameters, wherein the predetermined imaging parameters are obtained from at least one previously acquired image data set. Computer-readable medium that afford functionality of the type defined by this method is also contemplated in conjunction with the present technique.

In accordance with further aspects of the present technique, a system for imaging is presented. The system includes an acquisition subsystem configured to acquire a first image data set from a patient. In addition, the system includes a processing subsystem in operative association with the acquisition subsystem and configured to determine imaging parameters, store the imaging parameters with the first image data set acquired via the acquisition subsystem, and retrieve the stored imaging parameters for use in a subsequent examination.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary diagnostic system, in accordance with aspects of the present technique;

FIG. 2 is a diagrammatic illustration of the ultrasound imaging system for use in the diagnostic system of FIG. 1;

FIG. 3 is a flow chart illustrating an exemplary process of storing imaging parameters, in accordance with aspects of the present technique; and

FIG. 4 is a flow chart illustrating another exemplary process of imaging, in accordance with aspects of the present technique.

### DETAILED DESCRIPTION

As will be described in detail hereinafter, methods of imaging and a system for imaging configured to enhance efficiency of serial studies is presented. Employing the methods and system described hereinafter, the system for imaging may be configured to adapt acquisition and processing protocols based on predetermined settings, thereby aiding a clinician in obtaining information related to interval changes in temporally sequential images of a patient and improving the possibilities of detecting important changes in pathology.

Although the exemplary embodiments illustrated hereinafter are described in the context of a medical imaging system, it will be appreciated that use of the diagnostic system in industrial applications is also contemplated in conjunction with the present technique.

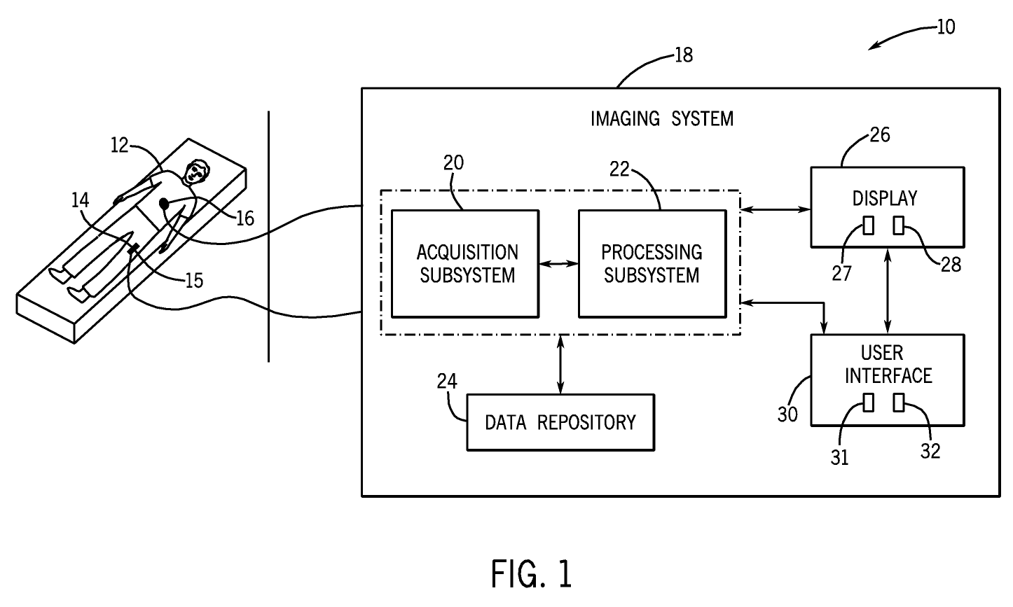

FIG. 1 is a block diagram of an exemplary diagnostic system 10 for use in diagnostic imaging in accordance with aspects of the present technique. The system 10 may be configured to acquire image data from a patient 12 via a probe 14, 16. Further, as used herein, “imaging” is broadly used to include two-dimensional imaging, three-dimensional imaging, or preferably, real-time three-dimensional imaging. Additionally, “imaging” may also include time-line modes such as spectral Doppler, M-mode and other functional imaging modes such as color flow or strain, to name a few. It should also be noted that although the embodiments illustrated are described in the context of a non-invasive or external probe 16, other types of probes, such as endoscopes, laparoscopes, surgical probes, transesophageal probes, transvaginal probes, transrectal probes, probes adapted for interventional procedures, including imaging catheters, for example, or combinations thereof, are also contemplated in conjunction with the present technique. For example, reference numeral 15 is representative of a portion of a catheter-based probe 14 that can be disposed inside the vasculature of the patient 12.

In any event, the probes 14, 16 may also be employed to aid in the acquisition of image data. Also, in certain other embodiments, image data may be acquired via one or more sensors (not shown) that may be disposed on the patient 12. By way of example, the sensors may include physiological sensors such as electrocardiogram (ECG) sensors and/or positional sensors such as electromagnetic field sensors or inertial sensors. These sensors may be operationally coupled to a data acquisition device, such as an imaging system, via leads (not shown), for example.

The system 10 may also include a medical imaging system 18 that is in operative association with the catheter-based probe 14 and/or the external probe 16. It should be noted that although the exemplary embodiments illustrated hereinafter are described in the context of a medical imaging system, other imaging systems and applications, such as industrial imaging systems and non-destructive evaluation and inspection systems, such as pipeline inspection systems and liquid reactor inspection systems, are also contemplated. Additionally, the exemplary embodiments illustrated and described hereinafter may find application in multi-modality imaging systems that employ ultrasound imaging in conjunction with other imaging modalities, position-tracking systems, or other sensor systems. Furthermore, it may also be noted that, although the embodiments illustrated are described in the context of an ultrasound imaging system, other types of imaging systems, such as a magnetic resonance imaging (MRI) system, an X-ray imaging system, a nuclear imaging system, a positron emission tomography (PET) system, or combinations thereof are also contemplated in conjunction with the present technique.

In a presently contemplated configuration, the medical imaging system 18 may include an acquisition subsystem 20 and a processing subsystem 22. Further, the acquisition subsystem 20 of the medical imaging system 18 may be configured to acquire image data representative of one or more anatomical regions of interest in the patient 12 via the catheter-based probe 14 and/or the external probe 16. The image data acquired from the patient 12 may then be processed by the processing subsystem 22.

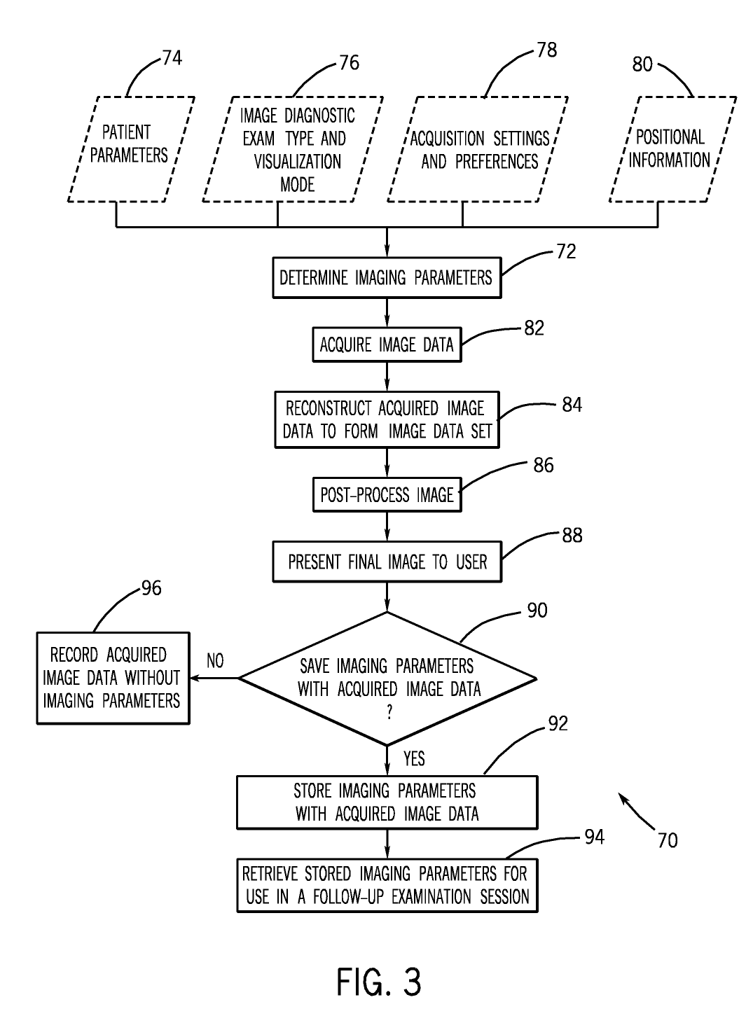

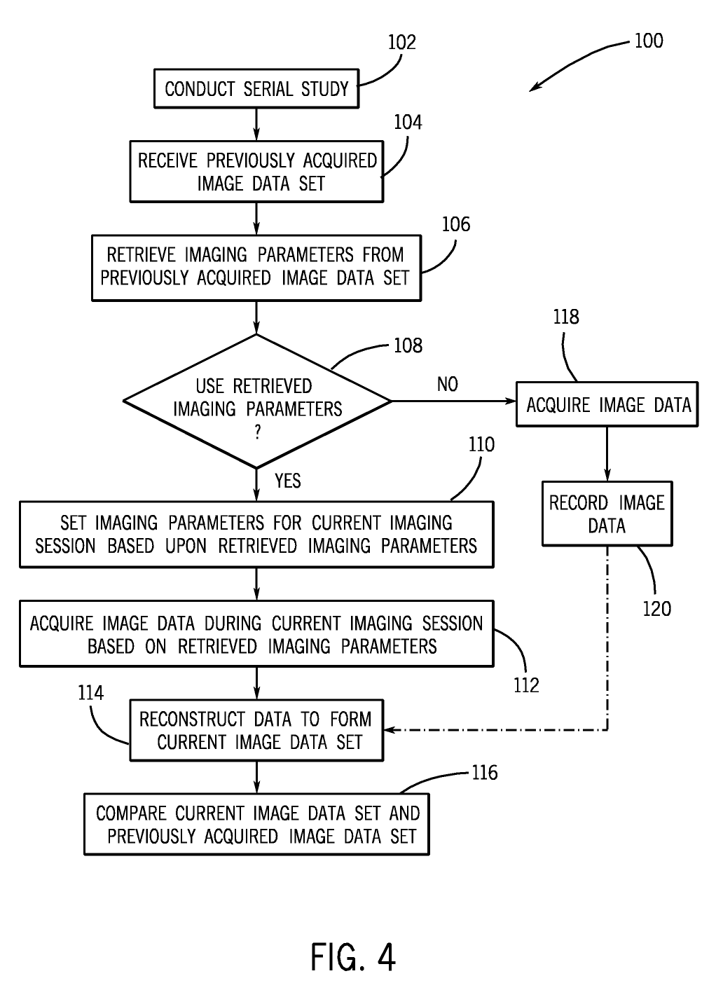

Additionally, the image data acquired and/or processed by the medical imaging system 18 may be employed to aid the clinician in identifying disease states, assessing need for treatment, determining suitable treatment options, and/or monitoring the effect of treatment on the disease states, as will be described in greater detail with reference to FIGS. 3-4. It may be noted that the terms treatment and therapy may be used interchangeably. In certain embodiments, the processing subsystem 22 may be further coupled to a storage system, such as a data repository 24, where the data repository is configured to receive ultrasound image data.

Furthermore, in certain embodiments, the catheter-based probe 14 may also be configured to deliver therapy to the identified one or more regions of interest. As used herein, “therapy” is representative of ablation, percutaneous ethanol injection (PEI), cryotherapy, and laser-induced thermotherapy. Additionally, “therapy” may also include delivery of tools, such as needles, for delivering gene therapy, for example. Additionally, as used herein, “delivering” may include various means of providing therapy to the one or more regions of interest, such as conveying therapy to the one or more regions of interest or directing therapy towards the one or more regions of interest. As will be appreciated, in certain embodiments the delivery of therapy, such as RF ablation, may necessitate physical contact with the one or more regions of interest requiring therapy. However, in certain other embodiments, the delivery of therapy, such as high intensity focused ultrasound (HIFU) energy, may not require physical contact with the one or more regions of interest requiring therapy.

As illustrated in FIG. 1, the medical imaging system 18 may include a display 26 and a user interface 30. However, in certain embodiments, such as in a touch screen, the display 26 and the user interface 30 may overlap. Also, in some embodiments, the display 26 and the user interface 30 may include a common area. In accordance with aspects of the present technique, the display 26 of the medical imaging system 18 may be configured to display an image generated by the medical imaging system 18 based on the image data acquired via the probe 14, 16. Additionally, the display 26 may be configured to aid the user in defining and visualizing image acquisition, as will be described in greater detail hereinafter. It should be noted that the display 26 may include a three-dimensional display. In one embodiment, the three-dimensional display may be configured to aid in identifying and visualizing three-dimensional shapes.

Further, the user interface 30 of the medical imaging system 18 may include a human interface device (not shown) configured to facilitate the user in identifying the one or more regions of interest for therapy using the image of the region displayed on the display 26. The human interface device may include a mouse-type device, a trackball, a joystick, a stylus, or a touch screen configured to facilitate the user to identify the one or more regions of interest requiring therapy. However, as will be appreciated, other human interface devices, such as, but not limited to, a touch screen, may also be employed.

With continuing reference to FIG. 1, reference numerals 27 and 28 are representative of icons disposed on the display 26, while keys disposed on the user interface 30 are represented by reference numerals 31 and 32. More particularly, reference numeral 27 is representative of an icon that may be configured to aid a clinician in saving imaging parameters with acquired image data. Further, the icon 28 may be configured to be indicative of availability of imaging parameters associated with a retrieved image data set. Similarly, the clinician may elect to save imaging parameters with image data by clicking on the key 31 disposed on the user interface 30 of the medical imaging system 18. Also, by clicking on the key 32, the clinician may choose to use the retrieved imaging parameters. The process of saving the imaging parameters by clicking on the icon 27, the key 31, or both, and the process of using retrieved imaging parameters by clicking on the icon 28, the key 32, or both, will be described in greater detail with reference to FIGS. 3-4.

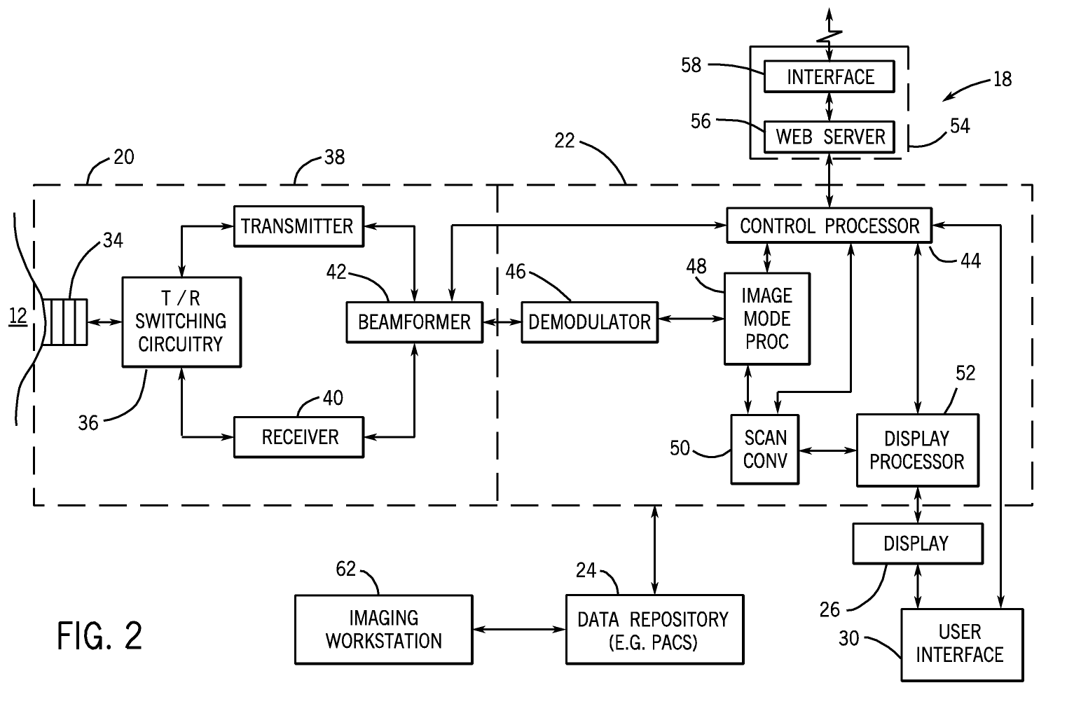

As previously noted with reference to FIG. 1, the medical imaging system 18 may include an ultrasound imaging system. Accordingly, FIG. 2 is a block diagram of an embodiment of an ultrasound imaging system 18 depicted in FIG. 1. The ultrasound system 18 in FIG. 2 is shown as including the acquisition subsystem 20 and the processing subsystem 22, as previously described. The acquisition subsystem 20 may include a transducer assembly 34. In addition, the acquisition subsystem 20 may include transmit/receive (T/R) switching circuitry 36, a transmitter 38, a receiver 40, and a beamformer 42. In one embodiment, the transducer assembly 34 may be disposed in the probe 14, 16 (see FIG. 1). Also, in certain embodiments, the transducer assembly 34 may include a plurality of transducer elements (not shown) arranged in a spaced relationship to form a transducer array, such as a one-dimensional or two-dimensional transducer array, for example. Additionally, the transducer assembly 34 may include an interconnect structure (not shown) configured to facilitate operatively coupling the transducer array to an external device (not shown), such as, but not limited to, a cable assembly or associated electronics. In the illustrated embodiment, the interconnect structure may be configured to couple the transducer array to the T/R switching circuitry 36.

The processing subsystem 22 may include a control processor 44, a demodulator 46, an imaging mode processor 48, a scan converter 50 and a display processor 52. The display processor 52 can be further coupled to a display 26 (see also FIG. 1) for displaying images. A user interface 30 (see also FIG. 1) interacts with the control processor 44 and the display 26. The control processor 44 may also be coupled to a remote connectivity subsystem 54 including a web server 56 and a remote connectivity interface 58. The processing subsystem 22 may be further coupled to the data repository 24 (see also FIG. 1) configured to receive ultrasound image data, as previously noted with reference to FIG. 1. The data repository 24 can also interact with an imaging workstation 62.

The aforementioned components may be dedicated hardware elements, such as circuit boards with digital signal processors, or they may be software running on a general-purpose computer or processor, such as a commercial, off-the-shelf personal computer (PC). The various components may be combined or separated according to various embodiments of the present technique. Thus, those skilled in the art will appreciate that the present ultrasound imaging system 18 is provided by way of example, and that the present techniques are in no way limited by the specific system configuration.

Referring to the acquisition subsystem 20, the transducer assembly 34 is in contact with the patient 12. The transducer assembly 34 is also coupled to the T/R switching circuitry 36. Also, the T/R switching circuitry 36 is in operative association with an output of the transmitter 38 and an input of the receiver 40. The output of the receiver 40 is an input to the beamformer 42. In addition, the beamformer 42 is further coupled to the input of the transmitter 38 and to the input of the demodulator 46. The beamformer 42 is also operatively coupled to the control processor 44, as shown in FIG. 2.

In the processing subsystem 22, the output of the demodulator 46 is in operative association with an input of the imaging mode processor 48. Additionally, the control processor 44 interfaces with the imaging mode processor 48, the scan converter 50, and the display processor 52. An output of the imaging mode processor 48 is coupled to an input of the scan converter 50. Also, an output of the scan converter 50 is operatively coupled to an input of the display processor 52. The output of the display processor 52 is coupled to the display 26.

The ultrasound system 18 transmits ultrasound energy into the patient 12 and receives and processes backscattered ultrasound signals from the patient 12 to create and display an image. To generate a transmitted beam of ultrasound energy, the control processor 44 sends command data to the beamformer 42 to generate transmit parameters to create a beam of a desired shape originating from a certain point at the surface of the transducer assembly 34 at a desired steering angle. The transmit parameters are sent from the beamformer 42 to the transmitter 38. The transmitter 38 uses the transmit parameters to properly encode transmit signals to be sent to the transducer assembly 34 through the T/R switching circuitry 36. The transmit signals are set at certain levels and phases with respect to each other and are provided to the individual transducer elements of the transducer assembly 34. The transmit signals excite the transducer elements to emit ultrasound waves with the same phase and level relationships. As a result, a transmitted beam of ultrasound energy is formed in the patient 12 along a scan line when the transducer assembly 34 is acoustically coupled to the patient 12 by using, for example, ultrasound gel. The process is known as electronic scanning.

In one embodiment, the transducer assembly 34 may be a two-way transducer. When ultrasound waves are transmitted into a patient 12, the ultrasound waves are backscattered off the tissue and blood samples within the patient 12. The transducer assembly 34 receives the backscattered waves at different times, depending on the distance into the tissue they return from and the angle with respect to the surface of the transducer assembly 34 at which they return. The transducer elements convert the ultrasound energy from the backscattered waves into electrical signals.

The electrical signals are then routed through the T/R switching circuitry 36 and to the receiver 40. The receiver 40 amplifies and digitizes the received signals and provides other functions, such as gain compensation. The digitized received signals corresponding to the backscattered waves received by each transducer element at various times preserve the amplitude and phase information of the backscattered waves.

The digitized signals are sent to the beamformer 42. The control processor 44 sends command data to the beamformer 42. The beamformer 42 uses the command data to form a receive beam originating from a point on the surface of the transducer assembly 34 at a steering angle typically corresponding to the point and steering angle of the previous ultrasound beam transmitted along a scan line. The beamformer 42 operates on the appropriate received signals by performing time delaying and focusing, according to the instructions of the command data from the control processor 44, to create received beam signals corresponding to sample volumes along a scan line within the patient 12. The phase, amplitude, and timing information of the received signals from the various transducer elements is used to create the received beam signals.

The received beam signals are sent to the processing subsystem 22. The demodulator 46 demodulates the received beam signals to create pairs of I and Q demodulated data values corresponding to sample volumes along the scan line. Demodulation is accomplished by comparing the phase and amplitude of the received beam signals to a reference frequency. The I and Q demodulated data values preserve the phase and amplitude information of the received signals.

The demodulated data is transferred to the imaging mode processor 48. The imaging mode processor 48 uses parameter estimation techniques to generate imaging parameter values from the demodulated data in scan sequence format. The imaging parameters may include parameters corresponding to various possible imaging modes, such as B-mode, color velocity mode, spectral Doppler mode, and tissue velocity imaging mode, for example. The imaging parameter values are passed to the scan converter 50. The scan converter 50 processes the parameter data by performing a translation from a scan sequence format to a display format. The translation includes performing interpolation operations on the parameter data to create display pixel data in the display format.

The scan converted pixel data is sent to the display processor 52 to perform any final spatial or temporal filtering of the scan converted pixel data, to apply grayscale or color to the scan converted pixel data, and to convert the digital pixel data to analog data for display on the display 26. The user interface 30 is also coupled to the control processor 44 to allow a user to interface with the ultrasound imaging system 18 based on the data displayed on the display 26.

Currently available transducer assemblies 34 typically include one or more transducer elements, one or more matching layers, and a lens. The transducer elements may be arranged in a spaced relationship, such as, but not limited to, an array of transducer elements disposed on a layer, where each of the transducer elements may include a transducer front face and a transducer rear face. As will be appreciated by one skilled in the art, the transducer elements may be fabricated employing materials, such as, but not limited to, lead zirconate titanate (PZT), polyvinylidene difluoride (PVDF), or composite PZT. The transducer assembly 34 may also include one or more matching layers disposed adjacent to the front face of the array of transducer elements, where each of the matching layers may include a matching layer front face and a matching layer rear face. The matching layers facilitate matching of an impedance differential that may exist between the high impedance transducer elements and a low impedance patient 12. The lens may be disposed adjacent to the matching layer front face and provides an interface between the patient 12 and the matching layer.

Additionally, the transducer assembly 34 may include a backing structure, having a front face and a rear face, which may be fabricated employing a suitable acoustic damping material possessing high acoustic losses. The backing structure may be acoustically coupled to the rear face of the array of transducer elements, where the backing structure facilitates the attenuation of acoustic energy that may emerge from the rear face of the array of transducer elements. In addition, the backing structure may include an interconnect structure. Moreover, the transducer assembly 34 may also include an electrical shield (not shown) that facilitates the isolation of the transducer elements from the external environment. The electrical shield may include metal foils, where the metal foils may be fabricated employing metals such as, but not limited to, copper, aluminum, brass, or gold.

Referring now to FIG. 3, a flow chart of exemplary logic 70 for storing imaging parameters is illustrated. In accordance with exemplary aspects of the present technique, a method for imaging one or more regions of interest in the patient 12 (see FIG. 1) is presented. The method starts at step 72, where imaging parameters are determined. As used herein, the term imaging parameters may be defined to include parameters, such as, but not limited to, patient parameters, user selected parameters, system acquisition parameters, positional information, or combinations thereof. Accordingly, in one embodiment, the determining step 72 may include acquiring patient parameters 74, receiving user selected parameters 76, selecting system acquisition settings 78, obtaining positional information 80, or combinations thereof, as depicted in FIG. 3. Although FIG. 3 shows the imaging parameters as including parameters such as patient parameters 74, user selected parameters 76, and system acquisition settings 78, use of other settings and parameters are also contemplated. By way of example, information related to the physiology of the patient 12 and/or pharmaceuticals may be employed. More particularly, information related to the physiology of the patient 12 and/or pharmaceuticals may include data indicative of an image being acquired after cardiac stress and/or a stress inducing agent. Similarly, the information may also include data representative of an image being obtained after a certain time interval past injection of a contrast agent, for instance. It may be noted that the imaging parameters may be set and/or changed by a clinician using the display 26 (see FIG. 1), the user interface 30 (see FIG. 1), or both.

In accordance with aspects of the present technique, patient parameters 74 may include patient information associated with a patient, such as the patient 12. The patient information may include the name of the patient, the patient's vital statistics, date of birth, social security number, and medical record number, to name a few. Additionally, the patient parameters 74 may include information regarding the anatomical region being imaged.

Furthermore, user selected parameters 76 may be representative of image diagnostic examination type, imaging mode, and/or visualization mode selected by the clinician. For example, the imagine mode may include two-dimensional imaging, three-dimensional imaging, real-time three-dimensional imaging, B-mode, M-mode, color velocity mode, spectral Doppler mode, tissue velocity imaging mode, and other functional imaging modes, such as color flow or strain, to name a few. Also, system acquisition parameters 78 may include a system identification number, a system model number, and/or revision numbers for the system 18, probes 14, 16, and/or software. Additionally, the system acquisition parameters 78 may include a desirable scan rate, a source filter material and thickness, a tube voltage, a current, a frequency setting, focal parameters, type of display, dynamic range, or combinations thereof.

It may be noted that other imaging parameters, such as, but not limited to, application preset, steering angle for acquired image, display depth, number of steering angles used for compound images, speckle reduction imaging level, low gray rejection level, edge enhance, persistence, color map, gray map, image rotation, frequency, line density, coded excitation selection, transmit focus depth and number of zones, display dynamic range compression, acoustic output power, B-mode image softener, suppression, additional near field focal zones, time between lines to allow for decay, mode, color flow, map compress, map, velocity scale, accumulation for peak velocity, baseline position of velocity scale, Wall filter velocity threshold, pulse repetition frequency, trace sensitivity, sweep speed for timeline, cycles to average in pulse Doppler spectral display, time resolution in spectral Doppler timeline, range gate, range gate position, and/or Doppler steering angles may also be employed.

In accordance with further aspects of the present technique, positional information 80 associated with the patient 12 being imaged may be obtained. Further, it may be noted that the positional information 80 is associated with an imaging volume relative to one or more reference points. The positional information 80 may include localization coordinates of the patient 12 and/or the anatomical region being imaged. For example, the positional information 80 may include the XYZ coordinates of an anatomical region being imaged. Additionally, in certain imaging modalities, the positional information may include coordinates such as X, Y, Z, roll, pitch, and/or yaw. In certain embodiments, positional information 80 may be obtained via one or more position sensors (not shown) disposed on the patient 12. The position sensors may include an electromagnetic field sensor or an accelerometer, for instance. Further, the positional information 80 may also include anatomical markers and/or comments associated with the anatomical region being imaged. The anatomical markers may be obtained via input by the clinician, in one embodiment. Also, the positional information 80 may include information regarding patient orientation, such as sagittal orientation or coronal orientation, for example.

Subsequently, as indicated by step 82, image data representative of an anatomical region of interest of the patient may be acquired by a data acquisition device, such as the medical imaging system 18 (see FIG. 1). As previously noted, the image data representative of the anatomical region of the patient 12 may be acquired via a probe 14, 16 (see FIG. 1). The image data may be acquired in real-time employing the probe 14, 16. In addition, mechanical means, electronic means, or combinations thereof may be employed to facilitate the acquisition of image data via the probe 14, 16. This acquisition of image data aids the clinician in identifying disease states, assessing the need for therapy in the anatomical region being imaged, and/or monitoring efficacy of therapy on the identified disease states.

It may be noted that the acquisition of image data at step 82 may be based upon any suitable imaging modality, typically selected in accordance with the particular anatomy to be imaged and/or the analysis to be performed. By way of example, as will be appreciated by one skilled in the art, the physical limitation of certain imaging modalities render them more suitable for imaging soft tissues as opposed to bone or other more dense tissue or objects. Moreover, the modality may be coupled with particular settings, also typically dictated by the physics of the system, to provide higher or lower contrast images, volume rendering, sensitivity, or insensitivity to specific tissues or components, and so forth. Finally, the image acquisition may be coupled with the use of contrast agents or other markers used to target or highlight particular features or areas of interest. In a CT system, for example, the image data acquisition of step 82 is typically initiated by an operator interfacing with the system 18 via the user interface 30 (see FIG. 1). Readout electronics detect signals generated by virtue of the impact radiation on a scanner detector, and the system 18 processes these signals to produce useful image data. However, as will be appreciated by one skilled in the art, image data may also be accessed from image acquisition devices, such as, but not limited to, a magnetic resonance imaging (MRI) system or X-ray devices. In addition, while the image acquisition devices mentioned hereinabove may be used to directly acquire image data from a patient 12, image data may instead include data from an archive site or data storage facility.

Following step 82, the acquired image data along with the imaging parameters may then be reconstructed to generate an image data set, at step 84. The reconstructed image data set may then be post-processed at step 86. The post-processing step 86 may include a three-dimensional reformatting of the image. In certain embodiments, the reconstructed image data set may be subject to a filtering process to reduce image noise. It should also be noted that the visualization preferences selected by the clinician prior to the acquisition of image data at step 82 may influence the acquisition and processing of the image data. A final image may then be presented to the clinician at step 88 in accordance with the visualization preferences selected by the clinician prior to the acquisition of data.

As previously noted, a diagnostic imaging system, such as diagnostic system 10 (see FIG. 1) is frequently used to aid in identifying disease and/or monitoring the effect of treatment on disease states. Serial studies are typically conducted where images of a region of interest in a patient are acquired periodically during treatment and compared. Unfortunately, the configuration of the imaging system may be quite complex and often varies from one examination to the next, especially if different clinicians are involved in the acquisition of the temporally sequential images. The inconsistencies in the duplication of examination settings may disadvantageously result in undesirable variations and/or mask true variations, thereby leading to missed detection and/or diagnosis. Hence, it is desirable to reproduce the examination settings as closely as possible to circumvent the possibilities of missed detection.

Accordingly, the exemplary method of imaging includes storing of imaging parameters associated with a given examination. As previously noted, storing the imaging parameters with the image data acquired during an examination advantageously aids the clinician in reproducing the examination settings during a subsequent examination session. Accordingly, the imaging system 18 may be configured to store the corresponding set of imaging parameters with the image data, in accordance with aspects of the present technique. In one embodiment, the set of imaging parameters may be automatically stored with the corresponding image data set. Alternatively, the imaging system 18 may be configured to store the imaging parameters in response to a trigger signal, in certain embodiments. Furthermore, in accordance with further aspects of the present technique, the imaging system 18 may be configured to present the clinician with an option of saving the imaging parameters with the acquired image data. It may be noted that the stored parameters may then be retrieved for use in a follow-up or other subsequent examination session.

As noted hereinabove, the imaging system 18 may be configured to store the imaging parameters with the corresponding image data acquired at step 82 in response to a trigger signal. The trigger signal may be configured to be indicative of a desire to store imaging parameters with an associated image data set. Accordingly, a verification check may be carried out at step 90 to verify if the trigger signal is received. In one embodiment, a hard key can also be configured by a clinician to be associated with storing imaging parameters. By way of example, a key, such as the key 31 (see FIG. 1), on the user interface 30 of the imaging system 18 may be configured to facilitate storage of the imaging parameters with the corresponding set of image data. The trigger signal may be generated in response to the clinician electing to save the imaging parameters with the acquired image data by selecting the key 31. Consequently, the image data may be stored with the corresponding set of imaging parameters, at step 92. Subsequently, the stored imaging parameters may be retrieved for use in a follow-up examination session, as indicated by step 94.

However, at step 90, if the trigger signal is not received, then the image data may be stored without the corresponding set of imaging parameters, as indicated by step 96. Furthermore, in accordance with aspects of the present technique, another hard key, such as a Print key on the user interface 30 of the imaging system 18, may be configured to be associated with the storage of only image data. In other words, the imaging system 18 may be configured to execute step 96 in response to the Print key being selected, thereby storing the image data without the associated imaging parameters.

In accordance with further aspects of the present technique, the clinician may be presented with an option of saving the image data along with the corresponding imaging parameters. Alternatively, the clinician may choose to save the acquired image data without the imaging parameters. In one embodiment, the imaging system 18 may be configured to save the imaging parameters with the corresponding acquired image data in response to a trigger signal. The trigger signal may be generated in response to the clinician electing to save the imaging parameters with the acquired image data, in certain embodiments. In one embodiment, the clinician may elect to save the image data along with the corresponding imaging parameters by selecting the icon 27 (see FIG. 1) disposed on the display 26 of the imaging system 18.

As noted hereinabove, in certain embodiments, the imaging parameters may be stored automatically or in response to the selection of the icon 27, the key 31, or both. Following this election at step 90, the acquired image data may be stored along with the associated imaging parameters, as depicted in step 92. In certain embodiments, the imaging parameters may be stored in a digital imaging and communications in medicine (DICOM) header associated with the acquired image data. Furthermore, it may be noted that the imaging parameters may be stored in private tags of the DICOM header that are dedicated to a particular imaging system, such as imaging system 18. Consequently, the stored imaging parameters may only be accessed by the clinician using the imaging system 18. As will be appreciated, DICOM is a common standard for storing and/or receiving scans in a caregiving facility, such as a hospital. The DICOM standard was created to facilitate distribution and visualization of medical images, such as CT scans, MRIs, and ultrasound scans. Typically, a single DICOM file contains a header that stores information regarding the patient, such as, but not limited to, the name of the patient, the type of scan, and image dimensions. It may be noted that the acquired image data along with the imaging parameters may be saved on a local hard drive, a local database, an external storage device, such as a compact disc (CD), or it may be transmitted over a network to a remote storage device, as needed and/or desired.

With returning reference to the decision step 90, if the clinician elects to save the acquired image data without the imaging parameters, the image data acquired at step 82 may be recorded as indicated by step 96. The acquired image data may be saved on a local hard drive, a local database, an external storage device, such as a compact disc (CD), or it may be transmitted over a network to a remote storage device, as previously described. Further, the acquired image data that has been stored without the corresponding imaging parameters may also be reconstructed, and followed by the post-processing 86 and presentation to the clinician 88. It may be noted that the method of imaging described hereinabove may be employed to acquire data representative of an image and/or data representative of an image volume.

As previously noted, serial studies are typically conducted to aid a clinician in the diagnosis of disease states and/or to monitor the effect of treatment on the disease states. Further, as will be appreciated, temporally sequential images are acquired periodically during treatment. It may be noted that the temporally sequential images are typically acquired via the same imaging modality at different points in time, where the imaging modality may include a CT imaging system, an X-ray imaging system, a MR imaging system, an ultrasound imaging system, an optical imaging system, a PET imaging system, a nuclear medicine imaging system, or combinations thereof, as previously noted. The clinician may then evaluate the efficacy of treatment on the identified disease state by comparing two or more temporally sequential images. However, as will be appreciated, the configuration of the imaging system can be quite complex and may therefore be difficult to duplicate from one examination to the next. The variations in the configuration of the imaging system may disadvantageously result in undesirable variations and/or masked true variations, thereby leading to missed detection and/or diagnosis. The method of imaging presented in FIG. 3 may advantageously be employed to aid in reproducing the examination settings from one examination to the next, thereby circumventing possibilities of missed detection.

Referring now to FIG. 4, a flow chart of exemplary logic 100 for imaging is illustrated. In accordance with exemplary aspects of the present technique, a method for imaging one or more regions of interest is presented. More particularly, a method of imaging that may be configured to facilitate a clinician identifying a disease state and/or monitoring the effect of a treatment on a disease state is presented. The method starts at step 102, where a serial study may be initiated. In other words, temporally sequential images of a patient, such as the patient 12 (see FIG. 1) may be obtained. As previously noted, the temporally sequential images of the patient 12 may be employed for review of interval changes occurring between two or more images corresponding to a given patient, where the images are produced at different points in time.

Accordingly, when the patient 12 arrives for a current examination, one or more images associated with previous examinations corresponding to the same patient 12 may be restored, as depicted by step 104. The previously acquired images may be restored from an archive site or data storage facility. Subsequently, imaging parameters associated with the previously acquired image data set that has been obtained at step 104 may be retrieved at step 106. As previously noted, in certain embodiments, the imaging parameters associated with the previous examination may be stored in the DICOM header corresponding to a previously acquired image data set. In a presently contemplated configuration, if the previously acquired image data set was stored along with corresponding imaging parameters, the imaging system 18 (see FIG. 1) may be configured to communicate information to the clinician that is indicative of the availability of the imaging parameters. Information regarding the availability of stored imaging parameters may be communicated to the clinician via an indicator, on the display 26, for example. In a presently contemplated configuration, the indicator may include the icon 28 (see FIG. 1) on the display 26. The clinician may then select the indicator to retrieve the imaging parameters from the previously acquired image data set. More particularly, the imaging parameters associated with the previously acquired image data set may be retrieved from the DICOM header corresponding to the previously acquired image data set, for instance. Additionally, the clinician may choose to retrieve the imaging parameters via use of the key 32 (see FIG. 1), in certain embodiments.

Following the retrieval of the imaging parameters associated with the previously acquired image data set, the clinician may elect to duplicate system settings for the current examination session based on the retrieved imaging parameters. Alternatively, the clinician may choose not to use the retrieved imaging parameters. Accordingly, a check may be carried out to verify if the retrieved imaging parameters are to be duplicated for the current examination session, as depicted by decision step 108. In accordance with aspects of the present technique, if the clinician elects to duplicate the system settings for the current examination session based on the retrieved imaging parameters, then the clinician may command the imaging system 18 to duplicate all the settings for the current examination session based on the retrieved imaging parameters at step 110. In one embodiment, the clinician may command the imaging system 18 to duplicate all the settings by selecting the key 32 on the user interface 30 of the imaging system 18. Alternatively, in certain other embodiments, the imaging system 18 may be configured to automatically duplicate the settings for the current imaging session based on the retrieved imaging parameters.

Once the system settings for the current examination session have been set based upon the retrieved imaging parameters, image data representative of the current examination session may be acquired, as indicated by step 112. It may be noted that the image data acquired at step 112 is obtained based upon the retrieved imaging parameters associated with one or more previously acquired image data sets.

As previously noted, anatomical markers that have been previously stored as part of the imaging parameters may be employed to aid the clinician in replicating the imaging conditions. More particularly, the anatomical markers may be used to provide graphical guidance to the clinician to duplicate acquisition position of the patient 12 in the current examination session. In other words, the orientation of the patient 12 and/or imaging plane may be changed based upon the stored anatomical markers. For example, previously acquired image data representative of a region of interest and the associated stored anatomical markers may be recalled. Subsequently, image data representative of the same region of interest may be acquired during the current examination session. The orientation of a data acquisition device, such as the probe 14, 16 (see FIG. 1), may be altered such that the image data corresponding to the anatomical region having the anatomical marker is obtained. Additionally, the patient 12 and/or the imaging plane may also be reoriented such that image data that includes the anatomical marker is obtained. In other words, the current imaging volume is aligned with a corresponding previously acquired imaging volume based upon the anatomical markers in the previously acquired image data. This process of reorienting the patient and/or imaging plane may be repeated for multiple (e.g., three) anatomical markers, in one embodiment.

Subsequently, the current imaging volume that has been aligned with the corresponding imaging volume in the previously acquired image data may be registered with a matching imaging volume in the previously acquired image data. As will be appreciated, the process of finding the correspondence between the contents of the images is generally referred to as image registration. In other words, image registration includes finding a geometric transformation that non-ambiguously links locations and orientations of the same objects or parts thereof in the different images. More particularly, image registration includes transforming the different sets of image data to a common coordinate space.

The acquired image data may then be reconstructed to form a current image data set at step 114. Further, as previously noted, post-processing algorithms may be applied to the current image data set and the current image data set may be prepared for presentation to the clinician, as previously described with reference to FIG. 3.

Subsequently, at step 116, the current image data set generated at step 114 may be compared with at least one previously acquired image data set to aid the clinician in monitoring the disease state and/or evaluating the effect of treatment on the disease state. As will be appreciated, at step 116, the clinician may compare at least two temporally sequential images manually, in one embodiment. Alternatively, the comparison step 116 may be automated, where the two or more images may be compared by employing computer aided detection (CAD) algorithms, for example.

With returning reference to the decision step 108, if the clinician does not elect to use the retrieved imaging parameters for the current examination session, image data associated with the current examination session may be acquired, at step 118, based on settings that may be different from the retrieved imaging parameters associated with at least one previously acquired image data set. Subsequently, the image data acquired at step 118 may be recorded at step 120. As previously noted, this image data set may be stored with or without the corresponding imaging parameters.

In accordance with further aspects of the present technique, the set of image data that has been acquired without the use of the retrieved imaging parameters and recorded at steps 118-120 may be reconstructed to form a corresponding image data set, at step 114. Subsequently, this image data set may be compared with the previously acquired image data set at step 116. Furthermore, imaging parameters corresponding to either the previously acquired image data set or the current image data set may be retrieved and applied to the other image data set. It may be noted that the method described hereinabove with reference to FIG. 4 may be employed for repeating acquisition of an image or of an imaging volume.

As will be appreciated by those of ordinary skill in the art, the foregoing example, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, such as C++ or Java. Such code, as will be appreciated by those of ordinary skill in the art, may be stored or adapted for storage on one or more tangible, machine readable media, such as on memory chips, local or remote hard disks, optical disks (that is, CDs or DVDs), or other media, which may be accessed by a processor-based system to execute the stored code. Note that the tangible media may comprise paper or another suitable medium upon which the instructions are printed. For instance, the instructions can be electronically captured via optical scanning of the paper or other medium, then compiled, interpreted, or otherwise processed in a suitable manner if necessary, and then stored in a computer memory.

The methods of imaging and the system for imaging described hereinabove dramatically enhance speed of procedural time taken to perform serial studies. Furthermore, efficiency of the serial studies that involve monitoring and evaluating different sets of image data acquired from the same patient at different points in time may be substantially enhanced as the different sets of data are acquired under similar imaging conditions. In other words, the efficiency of the procedure is greatly improved as undesirable variations due to dissimilar imaging conditions is substantially reduced, thereby resulting in increasing diagnostic confidence and treatment accuracy.

In accordance with the foregoing, a technical effect is to acquire a current image data set from a patient based on predetermined imaging parameters from at least one previously acquired image data set. Another technical effect is to set system acquisition parameters for a current examination based on retrieved imaging parameters from a previous examination.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method of imaging, the method comprising:
determining imaging parameters;
acquiring image data from a patient;
storing the imaging parameters with the acquired image data; and
retrieving the stored imaging parameters for use in a subsequent examination.

2. The method of claim 1, wherein the determining step comprises at least one or more of:
acquiring patient parameters;
receiving user selected parameters; or
selecting system acquisition settings.

3. The method of claim 2, further comprising obtaining positional information about the patient.

4. The method of claim 2, wherein the patient parameters are representative of at least one of patient information or anatomy being imaged.

5. The method of claim 2, wherein the user selected parameters are representative of at least one of an image diagnostic examination type, imaging mode, or visualization mode.

6. The method of claim 2, wherein the system acquisition settings comprise at least one of a desirable scan rate, a source filter material and thickness, a tube voltage, a current, or combinations thereof.

7. The method of claim 1, further comprising:
reconstructing an image using the acquired image data to generate a reconstructed image;
applying post-processing algorithms to the reconstructed image to generate a final image; and
presenting the final image to a user.

8. The method of claim 7, further comprising storing the reconstructed image, wherein the reconstructed image comprises the acquired image data and the stored imaging parameters.

9. The method of claim 1, wherein the storing step comprises storing the imaging parameters in a corresponding digital imaging and communications in medicine (DICOM) header.

10. A method of imaging, the method comprising:
acquiring a current image data set from a patient based on predetermined imaging parameters, wherein the predetermined imaging parameters are obtained from at least one previously acquired image data set.

11. The method of claim 10, further comprising:
receiving the at least one previously acquired image data set;
retrieving the imaging parameters from the at least one previously acquired image data set; and
setting system acquisition parameters for a current examination session based on the retrieved imaging parameters.

12. The method of claim 10, further comprising comparing the current image data set and the at least one other previously acquired image data set.

13. The method of claim 10, wherein the current image data set and the at least one other previously acquired image data set are acquired via a same imaging modality at different points in time.

14. The method of claim 10, wherein each of the current image data set and the at least one other previously acquired image data set is acquired via an imaging system, wherein the imaging system comprises one of a computed tomography imaging system, a positron emission tomography imaging system, a magnetic resonance imaging system, an X-ray imaging system, an ultrasound imaging system, or combinations thereof.

15. A computer-readable medium comprising one or more tangible media, wherein the one or more tangible media comprise:
code adapted to acquire a current image data set from a patient based on predetermined imaging parameters, wherein the predetermined imaging parameters are obtained from at least one previously acquired image data set.

16. The computer-readable medium, as recited in claim 15, further comprising:
code adapted to receive the at least one previously acquired image data set;
code adapted to retrieve the imaging parameters from the at least one previously acquired image data set; and
code adapted to set system acquisition parameters for a current examination session based on the retrieved imaging parameters.

17. A system for imaging, comprising:
an acquisition subsystem configured to acquire a first image data set from a patient;
a processing subsystem in operative association with the acquisition subsystem and configured to:
determine imaging parameters;
store the imaging parameters with the first image data set acquired via the acquisition subsystem; and
retrieve the stored imaging parameters for use in a subsequent examination.

18. The system of claim 17, wherein the processing subsystem is further configured to:
reconstruct an image using the first image data set to generate a reconstructed image;
apply post-processing algorithms to the reconstructed image to generate a final image; and
present the final image to a user.

19. The system of claim 17, wherein the system is further configured to acquire at least a second image data set from the patient based on predetermined imaging parameters, wherein the predetermined imaging parameters are obtained from the first image data set.

20. The system of claim 19, wherein the system is further configured to compare the first image data set and at least the second image data set.

21. The system of claim 19, further comprising a display module configured to display the first image data set, the second image data set, or both.

22. The system of claim 17, wherein the system is further configured to:
receive the first image data set;
retrieve the imaging parameters from the first image data set; and
set system acquisition parameters for a current examination session based on the retrieved imaging parameters.
